(19) United States
(12) Patent Application Publication
(10) Pub. No.: US 2015/0247199 A1
(43) Pub. Date: Sep. 3, 2015

(54) LASER REJUVENATION

(71) Applicant: Ellex Medical Pty Ltd, Adelaide (AU)

(72) Inventors: Erica L. Fletcher, Parkville (AU); Victor Previn, Adelaide (AU)

(73) Assignee: Ellex Medical Pty Ltd.

(21) Appl. No.: 14/634,653

(22) Filed: Feb. 27, 2015

(30) Foreign Application Priority Data
Feb. 28, 2014 (AU) 2014900671

(51) Int. Cl. C12Q 1/68; A61B 5/00; A61B 3/10

(52) U.S. Cl. CPC C12Q 1/6883; A61B 3/1005; A61B 5/4848; C12Q 2600/16; C12Q 2600/118; C12Q 2600/158

(57) ABSTRACT

A method of treating a disease, disorder or condition of the eye includes irradiating the eye or eyes with a laser and subsequently detecting one or more biological responses by the eye or eyes to thereby determine the effectiveness of the laser treatment. The one or more biological responses by the eye or eyes are responses by cells, tissues and/or molecules of the eye or eyes that indicate, are associated with, or are functionally linked to, the effectiveness of the laser treatment. This may include detecting or measuring a change in the expression of one or more genes after laser treatment, such as cytoskeletal genes, matrix metalloproteinase genes, cell cycle genes and extracellular matrix genes. The method may include detecting or measuring cell differentiation, cell death and/or cell proliferation after laser treatment.

### FIELD OF THE INVENTION

This invention relates to laser treatments of the eye. More particularly, this invention relates to a laser treatment of the eye or eyes and measuring changes in gene expression as an indication of the effectiveness of the laser treatment.

### BACKGROUND TO THE INVENTION

Bruch's membrane is a multilayered, extracellular matrix that functions as a barrier to neovascularization while being permeable to small molecules such as oxygen and glucose. Bruch's membrane encircles more than half the eye and stretches with the corneoscleral envelope as intraocular pressure (IOP) increases. It therefore withstands this stretch and returns to its original shape when IOP decreases. This tissue also stretches to accommodate changes in choroidal blood volume. Finally, the choroid (and Bruch's membrane with it) may act as a spring that pulls the lens during accommodation. For these reasons, Bruch's membrane requires elasticity. The choroid also services the metabolic needs of the outer retina, facilitated in part by fenestrated endothelium. Oxygen, electrolytes, nutrients, and cytokines destined for the retinal pigmented epithelium (RPE) and photoreceptors pass from the choriocapillaris and through Bruch's membrane and waste products travel back in the opposite direction for elimination. Vitamins, signaling molecules and other factors needed for photoreceptor function are carried to the RPE by lipoprotein particles passing through Bruch's membrane, as do the RPE-produced lipoproteins that are eliminated in the opposite direction. The RPE pumps water from the sub retinal space to counter the swelling of the interphotoreceptor matrix GAGs. This fluid also flows across Bruch's membrane to reach the circulation.

Bruch's membrane is composed of five (5) layers. The RPE basal lamina is a meshwork of fine collagenous fibers, particularly containing collagen IV α3-5. The RPE synthesizes specific laminins that preferentially adhere Bruch's membrane to the RPE through interaction with integrins. The inner collagenous layer (ICL) contains fibers of collagens I, III, and V in a multilayered crisscross, parallel to the plane of Bruch's membrane. This collagen grid is associated with interacting molecules, particularly the negatively charged proteoglycans chondroitin sulfate and dermatan sulfate. The elastic layer (EL) consists of stacked layers of linear elastin fibers, crisscrossing to form a sheet with interfibrillary spaces. This sheet extends from the edge of the optic nerve to the ciliary body pars plana. In addition to elastin fibers, the EL contains collagen VI, fibronectin, and other proteins, and collagen fibers from the ICL and outer collagenous layer (OCL) can cross the EL. The EL confers biomechanical properties, vascular compliance and antiangiogenic barrier functions. The outer collagenous layer (OCL) contains many of the same molecular components as the ICL and the collagen fibrils running parallel to the choriocapillaris additionally form prominent bundles. This layer, unlike the ICL, has periodic outward extensions between individual choriocapillary lumens called intercapillary pillars. The choriocapillaris basal lamina is discontinuous with respect to Bruch's membrane due to the interruptions of the intercapillary pillars of the choroid. It is continuous with respect to the complex network of spaces defined by the choriocapillary lumens because the basal lamina envelops the complete circumference of the endothelium. A structural feature of the adjacent choriocapillary endothelium is fenestrations that are permeable to macromolecules. This basal lamina may inhibit endothelial cell migration into Bruch's membrane.

Bruch's membrane undergoes significant age-related changes that can result in the progression of AMD. Bruch's membrane thickens throughout adulthood two-to threefold under the macula (e.g. typically from about 2 μm to about 4.5 μm), becoming more variable between individuals at older ages. Equatorial Bruch's membrane changes little while Bruch's membrane near the ora serrata increases two-fold or more during this time. In the macula, the OCL thickens more prominently than the ICL. Unbalanced regulation of extracellular matrix molecules and their modulator matrix are thought to result in Bruch's membrane thickening. Increased histochemical reactivity for glycoconjugates, glycosaminoglycans, collagen and elastin is seen in the macula relative to the equator and near the ora serrata.

Along with thickening of Bruch's membrane, drusen accumulates between Bruch's membrane and the RPE and the presence of significant drusen in the macula are indicators of AMD. Drusen are made up of material such as apolipoproteins, complement proteins and relatively high levels of zinc, probably at least partly derived from the RPE and the choroid.

### OBJECT OF THE INVENTION

It is an object of this invention to provide a laser treatment for one or more diseases, disorders or conditions of the eye, wherein one or more biological responses of the eye are subsequently measured or detected to provide an indication of the effect of the laser treatment.

It is another object of this invention to provide a method whereby one or more biological responses of the eye or eyes are measured or detected to provide an indication of the effect of a laser treatment.

Further objects will be evident from the following description.

### DISCLOSURE OF THE INVENTION

In one form, although it need not be the only or indeed the broadest form, the invention resides in a method of determining the effect of a laser treatment of the eye or eyes, said method including the step of detecting one or more biological responses by the eye or eyes subsequent to laser treatment of the eye or eyes to thereby determine the effect of the laser treatment.

In another form, the invention resides in a method of preventing or treating a disease, disorder or condition of the eye including the step of irradiating the eye or eyes with a laser and subsequently detecting one or more biological responses by the eye or eyes as an indication of the effect of the laser treatment.

The one or more biological responses by the eye or eyes may include responses by cells, tissues and/or molecules of the eye or eyes that indicate, are associated with, or are functionally linked to, the effectiveness of the laser treatment.

In some embodiments, the methods of the aforementioned aspects may include the step of detecting or measuring a change in the expression of one or more genes after laser treatment.

In alternative or additional embodiments, the methods of the aforementioned aspects may include the step of detecting or measuring cell differentiation, death (e.g. apoptosis) and/or proliferation after laser treatment.

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

To assist in understanding the invention preferred embodiments will now be described with reference to the following figures in which:

FIG. 1 is a block diagram of a laser device;

FIG. 2 shows drusen resolution following nanosecond laser treatment. Multimodal image analysis was performed on a 74-yr-old woman with intermediate AMD using a Cirrus SD-OCT. Images were taken of the left eye before (A, C, E, G) and 12 mo after (B, D, F, H) nanosecond laser treatment. Color fundus photographs were taken (A, B) and the extent of drusen (pale yellow deposits in central retina, A) imaged using OCT (C, D), a segmented RPE map (E, F) and a drusen elevation heat map (G, H). At 12 mo following laser treatment, there was a substantial reduction in the extent of drusen in this participant. When the full cohort was analyzed for the proportion of individuals showing a reduction in drusen area (I), there was a significant reduction at 12 and 24 mo compared with a nature history cohort. When FAF images were assessed in regions of drusen resolution and compared with background areas within the same macula, most individuals (75%) showed stable FAF images, and the remaining individuals showed either increased or decreased autofluorescence, indicative of RPE/photoreceptor dysfunction and or loss (J). For autofluorescence data (J), n=12. Error bars represent the upper 95% confidence interval; n>43 individuals analyzed using a X² test. *P=0.05, **P<0.01, ***P<0.001.

FIG. 3 shows human and mouse retinal structure following nanosecond laser treatment. An 83-yr-old patient had 6 clinically relevant (0.3 mJ) and 6 suprathreshold (0.6 mJ) (arrows) nanosecond laser spots delivered to the superior and inferior macula respectively (A, fundus photo). After 5 d, the eye was surgically removed, immediately fixed and areas of retina corresponding to the clinical (C box) and suprathreshold (D box) doses were sectioned. Retinal structure was assessed using the cone photoreceptor marker, PNA (red) and calbindin (green), which labels numerous cell types in the human retina. Neither the clinical (C) nor suprathreshold (D) treatments altered retinal structure when compared with retinal areas disparate from the laser treatment sites (B); however, cells were observed in close association with the photoreceptor outer segments at the laser site (arrows). Mouse eyes were treated with 0.065 mJ and 0.13 mJ laser doses (E, fundus photo) and although the lower dose (G) produced no alteration in retinal structure compared with untreated retina (F), the higher dose led to significant disruption in the outer retina (H). Retinal cell death was assessed using TUNEL (red) 24 h following laser treatment in the low- (I) and high-energy (J) cohorts, and sections were counterstained with the nuclear label, DAPI (blue). The extent of TUNEL was quantified showing the 0.13 mJ dose resulted in photoreceptor cell death (K). INL, inner nuclear layer; GCL, ganglion cell layer. Quantitative data are presented as mean±SEM, n=3 analyzed using a 1-way ANOVA. **P<0.01. Scale bar, 50 μm.

FIG. 4 shows the integrity of human and mouse RPE following nanosecond laser treatment. Five days following nanosecond laser treatment, fixed human retina was flat mounted and the RPE imaged using phalloidin (red), which labels filamentous actin. Normal human RPE morphology (A) has a polygonal appearance, and the clinical dose induced a disrupted RPE monolayer (B). The RPE lesion was more defined after the suprathreshold laser treatment (0.6 mJ, C). Similar to the human, the 0.065 mJ energy treatment of the mouse retina produced a diffuse injury (E) compared with the untreated RPE (D), and the high dose (0.13 mJ) produced a more defined injury (F). One week after nanosecond laser treatment, the treated region in the human showed the presence of larger RPE cells at the injury border, with some cells extending into the lesion site (G), and after 1 mo the RPE monolayer was intact (H). Seven days post-treatment, the mouse RPE monolayer was reformed with enlarged cells covering the laser-treated site (I). This healing process and coincident increase in RPE cell size is quantified (J, K, respectively). Twenty-four hours after nanosecond laser (0.065 mJ), mouse RPE cells on the border of the laser treatment site (circles) were positive for cyclin-D1 (red, marker of cell proliferation), while RPE cells distant from the laser spot showed no indication of cyclin-D1 expression (L). Quantitative data presented as mean±SEM; n>3 analyzed using a 2-tailed Student's t test. **P<0.01. Scale bar, 100 μm.

FIG. 5. Shows the microglial response in human and mouse retina following nanosecond laser treatment. The human retina was sectioned through the clinically relevant nanosecond laser-treated area and stained with the cone photoreceptor marker, PNA (red); calbindin (blue), which labels numerous cell types in the human retina; and the mononuclear marker, ionized calcium-binding adapter molecule 1 (green). Five days after treatment, cells of mononuclear origin (green) are evident on the distal regions of the outer segments of the photoreceptors (A). In addition, retinal microglial cell processes are evident extending toward the laser-treated area. A similar microglial response is observed in the mouse retina 24 h following 0.065 mJ laser treatment (B, microglia in green, PNA in red). Three-dimensional reconstruction of the microglial response in the 0.065 mJ-treated mouse retina shows microglial processes extend toward the injury site (inset in B, microglia in green, PNA in red). The extension of microglial processes into the outer retina toward the injury site was quantified (C). When exposed to low-energy laser dose, retinal microglia were not active (D), as a number of their morphologic features common to activation were unchanged (F). The high-energy laser treatment (0.13 mJ) resulted in an activated microglial phenotype (E). Control (G) and low-energy-treated mouse retina (0.065 mJ, H) had a similar expression of C3 (red; microglia green), with it confined to the retinal blood supply. High-energy laser treatment (0.13 mJ; I) resulted in extensive C3 expression and migration of mononuclear cells into the outer retina (see arrows in I). All mouse vertical sections were counterstained with the nuclear label, DAPI (blue). OS, outer segment; INL, inner nuclear layer; GCL, ganglion cell layer. Quantitative data presented as mean±SEM; n>4, analyzed using a 2-tailed Student's t test (C) and a 2-way ANOVA. **P<0.01. Scale bar, 50 μm;

FIG. 6 shows the effect of nanosecond laser treatment on BM thickness and RPE gene expression. ApoEnull and C57BL/6J mice (10 mo of age) were treated with nanosecond laser (20 spots at 0.065 mJ) and tissue taken and fixed after 3 mo. Representative micrographs for C57BL/6J fellow eye (A), C57BL/6J treated eye (B), ApoEnull fellow eye (C), and ApoEnull treated eye (D) are shown. BM thickness was quantified (E) and showed ApoEnull animals to have a thickened membrane compared with C57BL/6J animals. Nanosecond laser treatment resulted in no significant alteration in BM thickness in the treated eye of C57BL/6J animals; however, there was a mean decrease. By contrast, the laser-treated eye of ApoEnull animals showed a significant reduction in membrane thickness. PCR array data showed 9 ECM genes were regulated >2-fold within the RPE samples from ApoEnull laser-treated eyes compared with samples from untreated ApoEnull control mice (F). The expression of genes encoding Mmp-2(G) and Mmp-3 (H) were quantified and both were reduced in the RPE samples from untreated ApoEnull animals, and laser treatment restored levels to those observed in the C57BL/6J animals. Ch, choroid. Quantitative data presented as mean±SEM; n>6, except in (F) where n=3. Data analyzed using a 2-way ANOVA. *P<0.05, **P<0.01, ***P<0.001. Scale bar, 1 μm.

FIG. 7 shows ApoEnull and C57BL6/6J mice (10 months of age) were treated with nanosecond laser (20 spots at 0.065 mJ) and tissue taken and fixed after 3 months. The expression of genes encoding retinal pigment epithelium protein 65 kDa (RPE-65) and Cathepsin D (Ctsd) as indicators of photopigment recycling (RPE-65) and lysosomal function (Ctsd) in the RPE. Both genes show no alteration in ApoEnull and C57BL6/6J control expression. Additionally, nanosecond laser treatment produced no alteration in gene expression in either the treated or fellow eyes in the ApoEnull and C57BL6/6J mice. Quantitative data are presented as mean±SEM; n>6).

FIG. 8 shows that laser irradiation of the eyes of mice with either 0.3 mJ, 0.8 mJ, 1.3 mJ or 1.8 mJ energy levels resulted in physical ablation of cells and demonstrates an initiation of repopulation of the RPE layer after 24 hours.

FIG. 9 shows that after laser irradiation at either 0.3 mJ, 0.8 mJ, 1.3 mJ or 1.8 mJ energy levels, labelling with vimentin antibodies and with alpha tubulin antibodies.

FIG. 10 shows that the Cyclin D1 marker of cell proliferation is induced in cells around the edge and within the laser-treated zones of the eye as time increases post laser ablation.

### SEQUENCE LISTING

The nucleic acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file named 94541-01SeqList.txt, created on Feb. 27, 2015, ˜4 KB, which is incorporated by reference herein.

### DETAILED DESCRIPTION

The invention is at least partly predicated on the discovery of cellular and molecular changes that occur in the eye or eyes post-laser irradiation. These cellular and/or molecular changes indicate the way in which the eye or eyes respond to laser treatment and therefore may provide a measure or indication of the success or effectiveness of the laser treatment. In this regard, it is proposed that changes in these cellular and/or molecular changes may be indicative of, associated with, or functionally linked to, the success or effectiveness of the laser treatment.

It will be appreciated that certain embodiments of the invention relate to the unexpected discovery that laser treatment of one or both eyes under conditions disclosed herein may result in a modulation or change in the expression of one or more genes.

As used herein a “gene” is a basic structural unit of a genome which includes a nucleotide sequence that encodes an amino acid sequence of a protein as well as other nucleotide sequences that perform regulatory or other non-coding functions, inclusive of 5′ untranslated sequences (e.g. a promoter), introns and 3′ untranslated sequences such as a polyadenylation sequence, although without limitation thereto.

The term “nucleic acid” is used broadly herein to include and encompass nucleotide sequences in the form of single- or double-stranded DNA, RNA and DNA:RNA hybrids, inclusive of mRNA and cDNA, although without limitation thereto. The term “nucleic acid” also includes probes, primers and oligonucleotides, as are well known in the art. In certain embodiments, nucleic acids such as probes, primers and oligonucleotides may be labeled (e.g. with fluorophores, digoxigenin, radionuclides, although without limitation thereto) to facilitate detection.

The term “protein” is used broadly herein to include and encompass any amino acid polymer inclusive of peptides and polypeptides. Constituent amino acids may be D- or L-amino acids, natural, non-natural or chemically modified amino acids

Changes in gene expression may be measured or expressed in relative or absolute terms and may encompass increases in gene expression and decreases in gene expression. In some embodiments, increases and/or decreases in gene expression may be measured or expressed in relation to a reference standard, such as a constitutively expressed gene. Gene expression may be measured at the level of nucleic acids such as RNA (e.g. mRNA) or cDNA or at the protein level. Detection and/or measurement of RNA may suitably performed using nucleic acid sequence amplification techniques as are well known in the art. Non-limiting examples of nucleic acid sequence amplification techniques include polymerase chanin reaction (PCR), ligase chain reaction, helicase-dependent amplification and Q-β replicase amplification, although without limitation thereto. Typically, nucleic acid sequence amplification includes the use of one or more primers that can anneal to a “target” nucleic acid to thereby specifically amplify the target nucleic acid.

Changes in gene expression may be detected or measured by hybridization-based techniques that typically utilize a labeled nucleic acid probe that is capable of specifically hybridizing to a target nucleic acid under appropriate stringency conditions. For example, RNA may be detected by hybridization-based techniques such as Northern blotting, in situ hybridization and by way of nucleic acid arrays, although without limitation thereto. These hybridization-based techniques may be performed alone or in combination with nucleic acid sequence amplification techniques that generate cDNA from RNA, the cDNA being subsequently detected by hybridization with a suitable nucleic acid probe that hybridizes to the cDNA “target” nucleic acid.

Proteins may be detected or measured by any protein-based technique known in the art. Advantageously, although not exclusively, protein detection may include the use of one or more antibodies or antibody fragments that specifically bind a protein of interest. Antibodies may be polyclonal or monoclonal, as is well understood in the art. Such techniques may include immunoblotting, immunoprecipitation, immunohistochemistry, ELISA and protein arrays such as proteomic arrays and tissue microarrays, although without limitation thereto. The antibody may be labeled (e.g. with biotin, digoxigenin, a fluorophore or an enzyme such as HRP or alkaline phosphatase) to facilitate detection or, alternatively, a secondary antibody may be so labeled.

Suitably, nucleic acid and protein detection is performed using a biological sample that comprises the nucleic acid(s) and/or protein(s) to be detected or measured. Biological samples may be in the form of nucleic acid extracts and protein extracts, inclusive of fluid, cell and tissue samples such as pathology samples, biopsies and ocular fluid samples such as sub retinal fluid samples and vitreous fluid samples, although without limitation thereto.

In certain embodiments, the genes may encode extracellular matrix proteins (“ECM genes”) inclusive of structural proteins, regulatory enzymes such as matrix metalloproteases (“MMP genes”) and cell-cell and cell-matrix interacting proteins such as set forth in Tables 2 and 3, although without limitation thereto. In other embodiments the genes may encode, cytoskeletal proteins (“cytoskeletal genes”) or cell-cycle proteins (“cell-cycle genes”), although without limitation thereto.

Non-limiting examples of MMP genes include Mmp2 and Mmp3 genes. In certain embodiments, expression of MMP genes is relatively increased following laser treatment. Increased expression may be observed up to 3 months after laser treatment.

Non-limiting examples of other ECM genes include collagen genes such as Col1a1, Col 5a1 and Col4a2, laminin genes such as Lama2, Lamb2, and Lamc1, genes that encode components of elastic fibers such as Emilin1 and genes that encode one or more integrin subunits. In certain embodiments, expression of one or more ECM genes is relatively increased following laser treatment.

Non-limiting examples of cytoskeletal genes include vimentin genes and tubulin genes. In one embodiment, increased expression of a cytoskeletal gene such as a vimentin gene indicates de-differentiation of epithelial cells to mesenchymal cells after laser treatment. In one embodiment, reduced expression of a cytoskeletal gene such as a vimentin gene indicates differentiation of mesenchymal cells to epithelial cells, such as RPE cells.

Non-limiting examples of cell-cycle genes include cyclin genes such as a cyclin D1 gene. In one embodiment, increased expression of a cell cycle gene such as a cyclin D1 gene indicates increased cell proliferation after laser treatment.

In some embodiments, the gene does not encode a heat shock protein (HSP) or ubiquitin.

In some embodiments, a cellular response may be measured or detected in response to laser treatment of the eye or eyes. As will be described in more detail in the Examples, following laser treatment laser epithelial cells such as RPE cells undergo ablation and an initial transition (i.e de-differentiation) to mesenchymal cells as part of an initial regeneration phase. This is accompanied by cell death (e.g. apoptosis) and cell proliferation. Finally, the mesenchymal cells differentiate to form new RPE cells in the eye or eyes.

It will therefore be appreciated that the invention provides molecular and cellular indicators that may be measured after laser treatment to thereby ascertain the effectiveness or success of the laser treatment.

The invention described herein may be broadly applied to the prevention or treatment of any disease or condition of the eye or eyes. These include degenerative diseases or conditions such as age-related macular degeneration (AMD), Diabetic Macular Edema (DME), glaucoma, retinitis pigmentosa, atrophy of the neuro-retina, stargardt disease, central serous retinopathy, choroidal neovascularization (CNV) and cystoid macular edema, although without limitation thereto.

In some embodiments, laser treatment may result in thinning of Bruch's membrane and/or a reduction of drusen level in the eye or eyes. In certain embodiments, the efficacy or progress of laser treatment as disclosed herein may be checked or monitored post-treatment by examining or measuring Bruch's membrane thickness and/or macular drusen levels. In this regard, a reduction in the thickness (i.e. “thinning”) of Bruch's membrane and/or a reduction in drusen levels would indicate that the laser treatment has been, or will be, successful.

The thickness of Bruch's membrane may be measured or detected by well known techniques such as optical coherence tomography (OCT) or computer-aided histological analysis such as described in Ramrattan et al., 1994, Invest. Opthamol. Vis. Sci 35 2857, although without limitation thereto.

Reduction in drusen levels may be subsequently measured or detected by well known techniques such as retinal imaging, optionally together with the use of suitable algorithms to quantify, correct, validate and/or model the results of retinal imaging.

Thickening of Bruch's membrane can occur with age and/or as an early symptom of AMD, DME, atrophy of the neuro-retina or CNV growth. Accordingly, it is proposed that in some embodiments laser treatment may be useful for reducing the thickness of Bruch's membrane to reverse or ameliorate the thickening that occurs as a result of ageing or the onset of AMD. This may improve the functioning of Bruch's membrane and thereby assist healing or repair of the RPE. Furthermore, a reduction in drusen levels may also be useful in treating AMD. In this context drusen “levels' may relate to drusen volume, surface area or any other measure of the amount of drusen. As will be described in more detail hereinafter, the thinning of Bruch's membrane and/or reduction in drusen levels may be associated with changes in gene expression, such as extracellular matrix genes and matrix metalloprotease genes, although without limitation thereto.

Accordingly, some embodiments of the invention relate to detecting or measuring thinning of Bruch's membrane and/or reduction in drusen levels in combination with measuring one or more cellular and/or molecular changes following laser irradiation of the eye or eyes as hereinbefore described.

Suitably, the laser is able to deliver the appropriate energy, duration and beam profile to affect the desired biological changes and avoid thermal collateral damage typical with some photocoagulator lasers.

A laser device suitable for performing the method of the invention delivers pulses of laser energy of relatively short duration and high peak power and at a wavelength that is preferentially absorbed by the treated cells tissues or organs. Typical laser parameters would be a laser pulse or sequence of laser pulses each having:

(A) a pulse duration in the range of 50 ps to 500 ns,

(B) a wavelength in the range 500 nm to 900 nm; and

(C) a pulse energy in the range 10 μJ to 10 mJ.

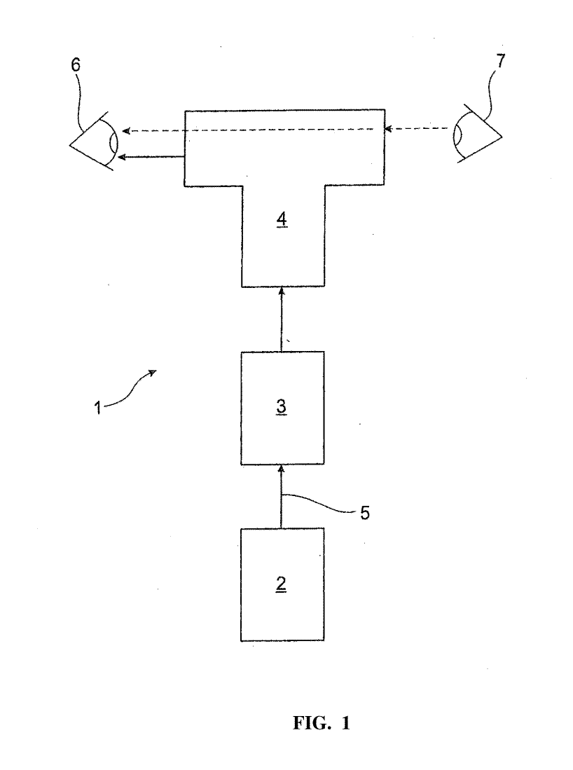

In particular embodiments relating to the treatment of the eye, where delicate cellular structures reside above the pigmented epithelium, such as the retina above the retinal pigmented epithelium (RPE), a preferred laser device is described in International Publication WO2008144828 and briefly with reference to FIG. 1.

In FIG. 1 there is shown a block diagram of a laser device 1. The laser device 1 comprises a laser module 2 producing a laser pulse or sequence of laser pulses each having a pulse duration in the range of 50 ps to 500 ns, a wavelength in the range 500 nm to 900 nm and a pulse energy in the range 10 μJ to 10 mJ. The laser device 1 further comprises a uniform irradiance module 3 that modifies an output beam profile of the laser module to produce a uniform treatment effect, and a beam delivery and viewing module 4 that delivers the laser pulse or pulses to the retina with a radiant exposure in the range of 8 mJ/cm² to 8000 mJ/cm² per pulse. A laser beam 5 is generated by the laser module 2, manipulated for uniform irradiance by the uniform irradiance module 3 and directed to the eye 6 of a patient by the beam delivery and viewing module 4. The beam delivery and viewing module 4 incorporates a coincident viewing path for an operator 7.

In use the laser device 1 irradiates the pigmented epithelium (PE) of the eye to produce intracellular micro-bubbles within the PE cells. The laser is optimized to deliver laser energy in a manner that causes rapid heating of the surface of pigmented granules within the PE cells, producing intracellular gas bubbles, but without sufficient time for the heat produced to diffuse through the outer cell membrane, thereby avoiding collateral damage. This is achieved by using pulses of laser energy of relatively short duration and high peak power, and at a wavelength that is preferentially absorbed by the RPE. Typically, radiant exposure is in the range 20 mJ/cm² to 300 mJ/cm² per pulse. The pulse duration is around 3 ns. The wavelength is suitably about 532 nm.

As described in WO2008/049164, when 3 ns pulses are used the first visible effect is from the formation of a macro-bubble, which results from intra-cellular micro-bubbles bursting the RPE cell membranes and coalescing into a visible macro-bubble. Therefore, radiant exposure levels no greater than 60-100% of the visible effect threshold (VET) are preferred to reduce the risk of damaging overlying photoreceptors. Particular values may be 60%, 65%, 70%, 75%, 80%, 85%, 90%, 95% or 100% of the VET.

Accordingly, the treatment radiant exposure may be titrated to a particular percentage below the VET in order to set the optimum value for each individual treated.

With regard to the invention disclosed herein, reference is made to the following non-limiting Examples.

### EXAMPLES

### Example 1

### Materials and Methods

### Human Studies

A nonrandomized prospective pilot clinical study was established (Australian New Zealand Clinical Trials Registry, ID: ACTRN12609001056280) to determine the effect of nanosecond laser treatment in the early stages of AMD. Fifty participants (50-75 yr) with bilateral intermediate AMD (drusen>125 μm; Ferris et al., 2013, Ophthalmology 120 844-851) and best corrected visual acuity of 20/63, were enrolled in the trial after written informed consent was obtained. After baseline examination, incorporating slit-lamp microscopic assessment, fundus examination, photography (Canon CR-DGi; Canon Incorporated, Tokyo, Japan), and spectral domain optical coherence tomography (SD-OCT; Cirrus; Carl Zeiss Meditec, Jena, Germany), the worst-performing eye was treated with a nanosecond, ultra-low-energy laser (3 ns, 2RT laser; Ellex, Adelaide, SA, Australia). Each patient received, in a single session, 12 laser spots (400 μm spot diameter) placed around the macula (>500 mm from the fovea), with energy levels individually titrated to be below the visual threshold for retinal change (range 0.15-0.45 mJ, average energy 0.24 mJ). In the current study, a natural history cohort was included for comparison of drusen resolution and consisted of 58 untreated subjects with AMD who had the same inclusion criteria as the treated group. All individuals were reviewed at 6 mo intervals for 2 yr.

In addition to the clinical trial, the effect of the nanosecond laser on the human retina/RPE was characterized in an 83-yr-old individual whose right eye was removed as part of an exenteration operation for an aggressive lid malignancy. Five days prior to exenteration, a full baseline examination was performed and multimodal retinal imaging performed. Six laser spots (400 μm diameter) were delivered to the superior macula just below the superior arcade at an energy of 0.3 mJ, which was considered the clinically relevant dose. An additional 6 suprathreshold spots (defined as 23 clinical dose, 0.6 mJ) were delivered to the inferior macula just above the inferior arcade. A second human eye was taken from an 84-yr-old individual who had been diagnosed with a lid basal cell carcinoma. Treatment was as described previously; however, nanosecond laser was performed at 2 time points, 1 mo and 1 wk prior to exenteration. Fundus photographs and OCT images were taken before and immediately after treatment as well as just prior to the surgery. For human studies, ethical approval was obtained from the Human Ethics Committee of the Royal Victorian Eye and Ear Hospital and studies were conducted in adherence with the Declaration of Helsinki.

### Animal Experiments

The effect of the nanosecond laser on mouse retinal structure was investigated using 3-mo-old C57BL/6J (general retinal structure) and Cx3cr1^{GFP/+} (on a C57BL/6J background; mononuclear cell response) animals. Cx3cr1^{GFP/+} animals have 1 copy of the microglial/macrophage-specific gene, Cx3cr1, replaced by the gene for enhanced green fluorescent protein (EGFP) (Jung et al., 2000, Mol. Cell. Biol. 20 4106-4114). These heterozygote animals are functionally normal, yet allow the morphology of microglial cells to be easily assessed.

All animals were anesthetized (ketamine: xylazine, 67:13 mg/kg), received additional corneal anesthesia (0.5%; Alcaine; Alcon Laboratories, Hunenberg, Switzerland), and were bilaterally dilated (1% atropine sulfate; Alcon Laboratories). The nanosecond laser was used to deliver 20 spots (each 0.065 mJ) around the optic nerve in 1 eye, with the untreated fellow eye serving as a “within animal” control. Laser energy was determined in a similar manner to that used for human clinical application (the dose below the visual threshold for a retinal response=pseudoclinical dose). A second cohort of animals received a 20 suprathreshold (0.13 mJ; 2× the pseudoclinical dose) spots, and untreated age-matched untreated control animals (C57BL/6J, Cx3cr1^{GFP/+}) were also included. The retinal fundus was imaged (Micron III; Phoenix Research Labs, Pleasanton, Calif., USA) before and after laser treatment. Animals were allowed to recover, and tissue was isolated at 1, 5, and 24 h and 7 d post-treatment.

The ApoEnull model of AMD was used to assess the efficacy of the nanosecond laser on reducing BM thickness, a key pathologic change observed in patients with AMD. ApoEnull animals have the apolipoprotein gene, ApoE, inactivated by gene-specific targeting (Piedrahita et al., 1992, Proc. Natl. Acad. Sci. USA 89 4471-4475) and were obtained from the Animal Resources Centre (Murdoch, WA, Australia). Animals were aged for 10 mo (n=18) after which time a subset was anesthetized as above and unilaterally treated with 20 laser spots (n=9, 0.065 mJ) and allowed to recover. A group of age-matched C57BL/6J animals was similarly treated. Tissue was isolated 3 mo post-laser treatment. All animals were housed at <40 lux, with access to food and water ad libitum. Animal experiments were approved by the University of Melbourne Animal Ethics Committee and adhered to the American Association of Vision Research and Ophthalmology standards for the ethical treatment of animals.

### Quantification of Drusen Resolution

Drusen area was determined by 2 masked graders, using gold standard methods of estimating drusen area based upon color fundus photographs (Complications of Age-Related Macular Degeneration Prevention Trial Research Group, 2006, Ophthalmology 113 1974-1986). Images centered on the fovea (3000 μm radius) were taken at baseline, 12 mo, and 24 mo, and the macular was divided into 5 concentric circles, with each circle divided into 4 quadrants. The percentage of drusen area was calculated within each of these regions with drusen area compared at different time points and classed as the same (drusen area±5% of baseline), better (decrease in drusen area>5%), or worse (increase in drusen area>5%) than baseline area. These results were subsequently reviewed by an independent grader, masked to the first results. The addition of the natural history cohort allows the efficacy of the laser treatment to be determined. This was not previously possible. The proportions of laser-treated and untreated fellow eyes with reduced drusen area were compared with that observed in the natural history. Any eyes that had progressed to late disease (geographic atrophy, choroidal neovascularization) were excluded from further analysis of drusen area (see Table 1) (Guymer et al., 2014, Olin. Experiment. Ophthalmol. 42 466-479).

Assessment of RPE Change within Areas of Drusen Regression

A subset of patients with AMD who had shown drusen regression after nanosecond laser treatment (n=12) had fundus autofluorescence (FAF) images assessed at baseline, 12 mo, and 24 mo (Spectralis HRA+OCT; Heidelberg Engineering, Heidelberg, Germany). FAF imaging principally measures the autofluorescence of bisretinoid compounds in the RPE, provides information on the physiologic state of the RPE/outer retina, and is a marker for AMD progression to geographic atrophy (Delori et al., 1995, Ophthalmol. Vis. Sci. 36 718-729; Schmitz-Valckenberg et al., 2009 Surv. Ophthalmol. 54, 96-117). Images were analyzed using the method of Toy et al. (Toy et al., 2013, Am. J. Ophthalmol. 156 532, e1). Briefly, baseline, color fundus images were registered and the 12 and 24 mo images subtracted from baseline (ImageJ, 1.43 Freeware; National Institutes of Health, Bethesda, Md., USA) (Thévenaz et al., 1998, IEEE Trans. Image Process. 727-41). Specific areas of drusen regression were identified, and these areas were superimposed onto the FAF images. The distribution of grayscale levels was calculated within the identified area and compared with background measures. A pixel was determined to have increased autofluorescence when it exceeded the mean background grayscale level by 3 SD. Similarly, when the level was 3 SD less than the mean background level, its intensity was decreased. The extent of auto-fluorescence was then determined for baseline, 12 mo, and 24 mo images and classified as increased, decreased, or no change (levels equal±5%).

### Tissue Processing, Immunohistochemistry, Histology

Following laser treatment, human eyes were exenterated (5 d and 1 mo post-treatment), the anterior section removed at the limbus, and the posterior eyecup immediately placed in 4% paraformaldehyde (in 0.1 M phosphate buffer, pH 7.4) for 5 h. Following fixation, the eyecup was placed in phosphate buffer, carefully dissected into multiple regions and cryoprotected (graded sucrose in 0.1 M phosphate buffer, 10%, 20%, 30%). Mouse eyes (C57BL/6J, Cx3cr1^{GFP/+}) were enucleated, and the posterior eyecup was isolated as above. Tissues were placed in 4% paraformaldehyde for 30 min, washed in 0.1 M phosphate buffer, and cryoprotected as above. Samples were embedded in Tissue-Tek (Microm International GmbH, Walldorf, Germany) optimum cutting temperature frozen and vertically sectioned (14 mm) onto polysine slides (Menzel-Glaser, Braunschweig, Germany).

Sections were processed for fluorescent immunohistochemistry as previously described (Vessey et al., 2012, Ophthalmol. Vis. Sci. 53 7833-7846; Jobling et al., 2013, Invest. Ophthalmol. Vis. Sci. 54 3350-3359). To investigate the effect of the nanosecond laser on retinal structure, human sections were coincubated with a rhodamine-conjugated peanut agglutinin (PNA; 1:250; Vector Laboratories, Burlingame, Calif., USA) to label cone photoreceptors and an antibody to calbindin (mouse anti-calbindin, 1:2000; Swant, Marly, Switzerland), which labels multiple cell types in the human retina (Eliasieh et al., 2007, Invest. Ophthalmol. Vis. Sci. 48 2824-2830). Sections were subsequently incubated with a goat anti-mouse Alexa Fluor 488 secondary antibody (1:500; Molecular Probes, Incorporated, Eugene, Oreg., USA). To assess mononuclear cell response, human sections were labeled with the mononuclear marker, ionized calcium-binding adapter molecule 1 (rabbit anti-lbA1, 1:1500; Wako Pure Chemical Industries, Richmond, Va., USA) and subsequently reacted with a goat anti-rabbit secondary antibody conjugated to Alexa Fluor 488 (1:500; Molecular Probes).

Cx3cr1^{GFP/+} sections were labeled with Alexa Fluor 488-conjugated anti-GFP (goat; Rockland Immunochemicals, Gilbertsville Pa., USA). Complement factor 3 (C3) expression was determined using a rat anti-C3 mAb (1:100; Abcam, Cambridge, United Kingdom) and a goat anti-mouse Alexa Fluor 594 (1:500; Molecular Probes). To determine cell death in the mouse retina following nanosecond laser treatment, TUNEL was performed as per manufacturers' instructions (Roche Diagnostics, Basel, Switzerland). All cell nuclei were counterstained with DAPI (0.2 mg/ml; Life Technologies, Carlsbad, Calif., USA).

Human and mouse tissues were also processed in whole mount, with the fixed retinae gently dissected away from the respective tissue sections/eyecups. For RPE structural analysis (human, C57BL/6J), RPE tissue samples were incubated over night at 4° C. with a conjugated antibody to the filamentous actin label, phalloidin (Alexa Fluor 633 Phaloidin, 1:200; Life Technologies), and whole-mount Cx3cr1GFP/+ retinae were incubated with Alexa Fluor 488-conjugated anti-GFP (Rockland Immunochemicals). All retinal sections and retinal/RPE whole mounts were mounted (Dako, Glostrup, Denmark), imaged using a confocal microscope (Meta or Pascal LSM-5, Zen software; Zeiss, Jena, Germany).

Microglial morphology was quantified at 1, 5, and 24 h and 7 d post-laser treatment, with Z-stack images isolated from the outer plexiform layer (OPL; 10 images per retina, n=5 retinae) around the optic nerve (<1750 μm diameter), which included the laser-treated areas. Cell morphology was assessed using Metamorph software (Molecular Devices, Sunnyvale, Calif., USA), and microglial process extension into the outer segment was quantified after 24 h (n=4, ImageJ) and 3-dimensional renderings produced via Imaris image software (Biplane, Zurich, Switzerland). RPE cell size and wound diameter were quantified at 1, 5, 24 h and 7 d, and the number of TUNEL-positive cells were quantified 24 h after laser treatment (n=3, per retinal area, ImageJ).

For retinal histology, posterior eyecups from C57BL/6J ani-mals were fixed overnight (1% paraformaldehyde, 2.5% glutaraldehyde, 3% sucrose, 0.01% calcium chloride in 0.1 M phosphate buffer, pH 7.4), washed in 0.1 M phosphate buffer, and then dehydrated in methanol (75, 85, 95, and 100%) and acetone (100%). Tissues were embedded (Epon resin; ProSciTech, Townsville City, QLD, Australia), polymerized, and sectioned (1 mm, Ultracut S; Reichert, Depew, NY, USA). Retinal sections were stained with 1% toluidine blue and imaged (Axioplan; Zeiss, Gottingen, Germany) (Vessey et al., 2011, J. Comp. Neurol. 519 506-527).

### Electron Microscopy

Transmission scanning electron microscopy was used to investigate RPE/BM structure in C57BL/6J and ApoEnull animals following nanosecond laser treatment. Posterior eye cups were isolated as above and fixed overnight (1% paraformaldehyde, 2.5% glutaraldehyde, 3% sucrose, 0.01% calcium chloride in 0.1 M phosphate buffer, pH 7.4). They were subsequently washed in 0.1 M cacodylate buffer pH 7.4, incubated for 1 h in 1% OsO4 in cacodylate buffer, dehydrated, and embedded (Epon resin). Ultrathin sections (70 nm) were cut and collected on formvar-coated copper grids. Sections were contrasted with uranyl acetate and lead citrate solutions, and viewed with a Phillips CM120 electron microscope (FEI, Hillsboro, Oreg., USA) (Vessey et al., 2012, supra). For analysis of BM thickness, 5-7 micrographs were taken for each ApoEnull and C57BL/6J animal in the control, laser treated, and untreated fellow eye cohorts (each n=6 per group). Membrane thickness measurements were taken at 5 separate locations within each image and subsequently averaged (at least 25 measures per animal).

### RNA Isolation and Quantitative PCR

For gene expression analysis, posterior eyecups from C57BL/6J and ApoEnull animals were isolated as described for immunohistochemical analysis. Retinae were removed from the posterior eyecup and snap frozen in liquid nitrogen. RPE-choroidal samples were isolated after the addition of 50 mL lysis buffer (RLTplus, RNeasy mini; Qiagen, Valencia, Calif., USA) containing 2-mercaptoethanol (1%), snap frozen in liquid nitrogen, and stored at −80° C.

Total RNA was isolated from the RPE-choroidal samples using commercial spin columns (RNeasy, Qiagen). A PCR gene array was used to assess the expression of 84 genes involved extracellular matrix (ECM) regulation (ECM and adhesion molecule array; Qiagen). Briefly, ApoE total RNA samples from untreated, laser-treated eyes and untreated fellow eyes (n=9 each group, each 25 ng) were pooled within their respective treatment groups (3 independent experiments containing 3 pooled samples), reverse transcribed (RT2 first strand; Qiagen) and then underwent preamplification of the cDNA target templates (RT2 pre-AMP; Qiagen). Samples were added to a commercial master mix (RT2 SYBR green master mix; Qiagen) and amplified for 40 cycles (ABI 7900HT; Life Technologies, Grand Island N.Y., USA). Three in-dependent arrays were performed for each treatment group. The data were analyzed using DDCt, expressed as fold change and regulation assessed using an unpaired t test.

Matrix metalloproteinase-2 and -3 (Mmp-2 and Mmp-3) gene expression was quantified in all treatment groups using quantitative PCR (n=9 each group). Total RNA samples from RPE/choroid (40 ng) were reverse transcribed (Sensiscript; Qiagen) and amplified (Rotor-Gene SYBR Green PCR kit; Qiagen) using specific primers (Mmp2: forward 5′-gtcgcccctaaaacagacaa-3′ (SEQ ID NO:1), reverse 5′-ggtctcgatggtgttctggt-3′ (SEQ ID NO:2); Mmp3: forward 5′-cagacttgtcccgtttccat-3′ (SEQ ID NO:3), reverse 5′-ggtgctgactgcatcaaaga-3′ (SEQ ID NO:4). External standards were used for quantification of gene copy number as previously described (Jobling et al., 2013, supra), using the housekeeping genes, hypoxanthine guanine phophoribosyl transferase (Hprt; forward 5′-cctaagatgagcgcaagttgaa-3′ (SEQ ID NO:5), reverse 5′-ccacaggactagaacacctgctaa-3′ (SEQ ID NO:6), and glyceraldehyde-3-phosphate dehydrogenase (Gapdh; forward 5′-tgtgtccgtcgtggatctga-3′ (SEQ ID NO:7), reverse 5′-ttgctgttgaagtcgcaggag-3′ (SEQ ID NO:8). Data were expressed as copies/copy Hprt.

### Statistical Analysis

In the pilot clinical trial, the proportion of laser-treated and untreated fellow eyes with reduced drusen area was compared with that observed in the natural history group using χ² test. All quantitative data from immunohistochemical and histologic studies were compared with untreated controls and assessed using t tests and 1- or 2-way ANOVAs where appropriate (see figure legends for specific test). Quantitative data from electron micrographs and real-time quantitative PCR were compared using a 2-way ANOVA and further assessed with a Bonferroni post hoc test.

### Results

Drusen Resolution in Patients with AMD Following Nanosecond Laser Treatment

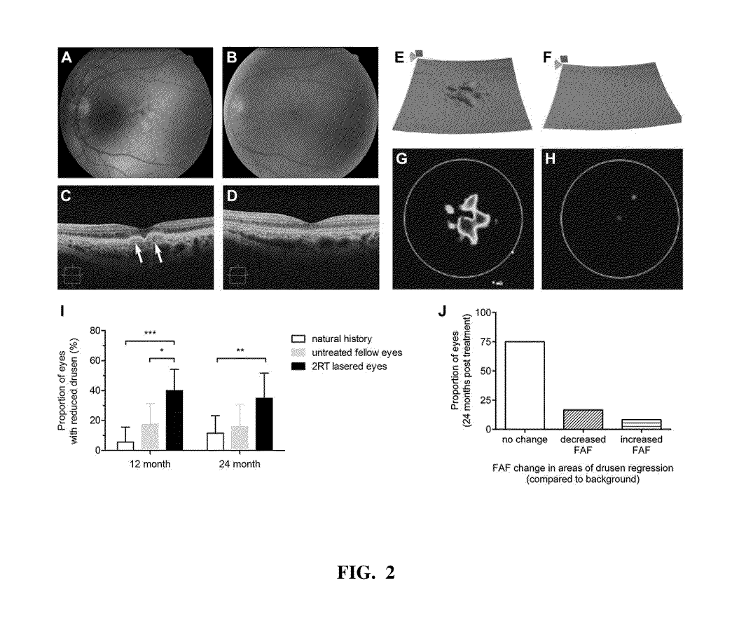

Sub-RPE drusen deposits are classic markers of AMD and are also used in the assessment of disease progression (Hageman et al., 2001, Prog. Retin. Eye Res. 20 705; Sarks, 1982, Aust. J. Opothalmol. 10 91). To determine the capacity of the nanosecond laser to reduce drusen in those with AMD, 50 individuals with intermediate AMD (drusen>125 μm) underwent baseline visual assessment and had 12 nanosecond laser spots (Ellex 2RT laser) delivered unilaterally to the macula. Multi-modal in vivo imaging was used to grade drusen change up to 2 y post-treatment. FIG. 1 shows the fundus of one participant pre-laser treatment (FIG. 2A) with distinct yellow drusen present in and around the macula. Using SD-OCT, these sub-RPE deposits can be observed in retinal cross section (see arrows in FIG. 2C), and the disruptive nature of drusen on the RPE monolayer is evident in the RPE contour (FIG. 2E) and heat (FIG. 2G, red=greater displacement) maps. When the same retinal area was reassessed 12 mo after nanosecond laser treatment, the fundus image showed a reduction in drusen number and area in and around the macula (B), and this regression was supported by the SD-OCT scan (FIG. 2D) and subsequent multimodal analysis (FIG. 2F, H).

Although several individuals showed complete resolution of drusen deposits similar to that shown in FIG. 2A-H, not all participants exhibited such complete resolution). To assess the cohort effect, drusen regression was quantified in the treated and untreated fellow eyes in the nanosecond laser-treated cohort and compared with an age-matched natural history cohort. After 12 mo, of those who had not reached a form of late disease (see Table 1), 40% of treated eyes had a reduction in drusen area in the treated eye compared with baseline measurements. The inclusion of the natural history cohort allows the efficacy of the treatment to be statistically validated. When compared, the percentage of eyes that showed a reduction at 12 mo was significantly greater than the 5% reduction observed in the natural history cohort (P<0.001). This positive effect was maintained at the 24 mo time point, with 35% of the treated eyes showing regression, and only 11% of eyes in the natural history cohort exhibited less drusen (P<0.01). Interestingly, the untreated fellow eye in the nanosecond laser-treated group also showed a regression in drusen compared with the natural history cohort at 12 mo (P=0.05); however, this effect was not significant at 24 mo (P>0.05).

Spontaneous drusen regression in AMD has been linked in some patients with disease progression, involving RPE atrophy and outer retinal change. To determine whether the RPE showed pathologic alterations in areas of drusen regression, FAF imaging was analyzed in a subset of laser-treated eyes 24 mo after treatment and compared with baseline images. Data indicated that 75% of eyes showed no alteration in autofluorescence in areas where drusen had resolved, and the remaining 25% of eyes showed either a decrease (16.7%) or increase (8.3%) in FAF readings compared with baseline after drusen regression (FIG. 2J). As decreased FAF is indicative of RPE cell loss (geographic atrophy) and increased FAF reflects lipofuscin accumulation (precedes RPE damage), any change in the autofluorescence profile would be suggestive of AMD progression. Thus, the nanosecond laser-induced reduction in drusen load appeared independent of the RPE change in the majority of patients, implying no indication of AMD progression to atrophy.

### Preservation of Retinal Structure Following Nanosecond Laser Treatment

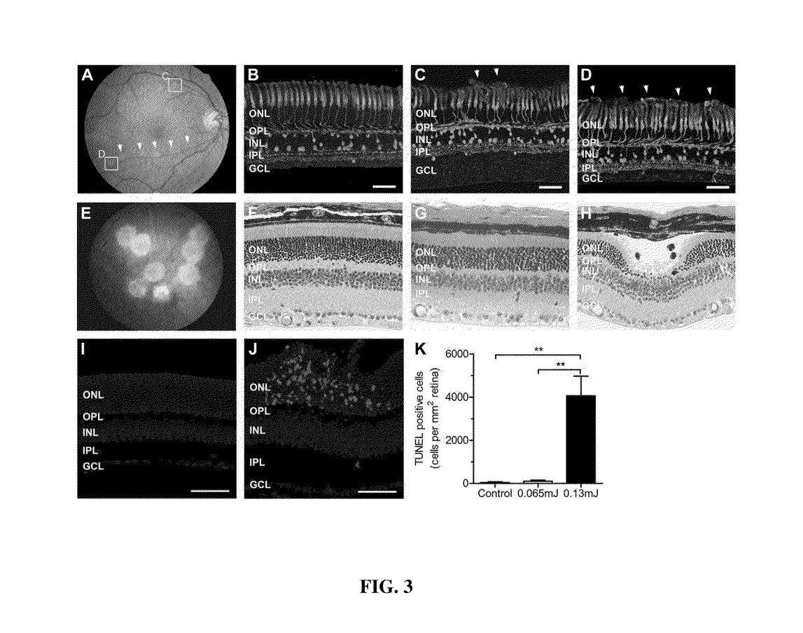

Traditional thermal ophthalmic lasers, although resolving drusen, also lead to neuronal injury and death in the overlying retina. In some cases, these treatments alter BM integrity, hastening disease progression (Olk et al., 1999, Opthalmology 106 2082). This collateral damage may reduce the effectiveness of lasers to limit AMD progression. Nanosecond laser treatment was performed on the right eye of an 83-yr-old individual 5 days prior to a scheduled exenteration procedure for the presence of an aggressive eyelid malignancy. Six laser spots were delivered to the macula just below the superior vascular arcade at 0.3 mJ (a clinically relevant dose), and 6 suprathreshold spots (0.6 mJ) were delivered to the macula, just above the inferior vascular arcade. The fundus photograph taken after treatment (FIG. 3A) shows a macula, with the suprathreshold spots evident as discrete bleached areas of retina (arrowheads and box D). The superior, clinically relevant laser doses are less clear upon fundus examination (box C).

Immediately after exenteration (5d post-laser treatment) the enucleated eye was fixed and vertical sections taken through the 0.3 mJ (box C in fundus, shown in FIG. 3C) and suprathreshold (box D in fundus, shown in FIG. 3D) laser-treated areas. Retinal structure was assessed using the cone photoreceptor marker PNA (red) and calbindin (green), which labels multiple neuronal cell types in the human retina. When compared with an untreated area of the retina (FIG. 3B), the area of the retina directly over the 0.3 and 0.6 mJ laser spots was unaffected, with no major structural change nor neuronal death evident (FIG. 3C, D, respectively). Of particular interest, the outer segments of the cone photoreceptors (outer region of the outer nuclear layer [ONL]) remain unaltered by the laser treatment, despite being situated directly over the laser-treated RPE. Yet although the outer segments show no indication of degeneration, accumulated cells can be seen in close contact with the tips of the outer segments within the treated region (see arrows in FIG. 3C, D).

Mouse models are particularly useful when investigating the mechanism or treatment of ocular diseases (Fletcher et al., 2011, Prog. Mol. Biol. Transl. Sci. 100 211-286). To determine whether mouse retina was equally resistant to the nanosecond laser and provide a basis for the use of this laser on a mouse model of AMD, adult C57BL/6J mice were treated with a 0.065 mJ laser dose to approximate the clinical condition in the human eye. It should be noted that although the same clinical criteria for determining laser energy dose was used in mouse experiments, the differences in dose (i.e., 0.065 mJ vs. 0.3 mJ) reflect the differences in pigmentation, eye size, and optics of a mouse eye compared with that of a human. As can be observed from the fundus photo (FIG. 3E), the 0.065 mJ dose produced a larger and more obvious bleached area within the mouse retina compared with the human, with this effect likely due to the greater degree of RPE pigmentation found in the C57BL/6J mouse eye. Despite this degree of bleaching, the retinal structure over the treated area remained unaffected when compared with an untreated retinal section (FIG. 3F vs. 3G). To determine whether nanosecond laser treatment could result in retinal damage, a higher laser dose was used (0.13 mJ). After this high-dose treatment, the mouse retina exhibited significant disruption, particularly in the ONL, with most of the cells in that layer lost (FIG. 3H). Retinal cell death (TUNEL, red) was assessed after the 0.065 mJ (FIG. 3I) and 0.13 mJ (FIG. 3J) treatments, with most of the cell death found in the high-energy treatment confined to the ONL (see FIG. 3J). When cell death was quantified (FIG. 3K), the 0.065 mJ laser dose produced no significant retinal cell death compared with the untreated control (100±52 vs 41±30 cells/mm², P>0.05), while numerous TUNEL-positive cells were present in the 0.13 mJ treatment (4064±907 cells/mm² retina, P<0.01). Thus, like in the human eye, low-energy nanosecond laser treatment does not lead to retinal damage in the mouse; however, when laser energies are significantly increased, retinal cell death can occur.

### Nanosecond Laser Treatment Targets the RPE

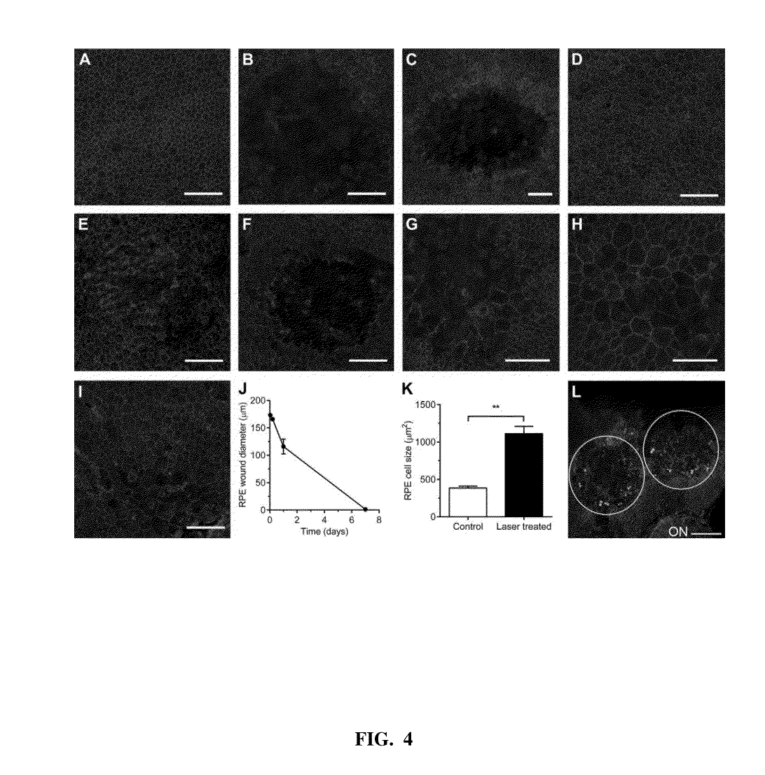

We addressed whether the nanosecond laser specifically targets the RPE monolayer underneath the retina in humans. Flat-mounted retinal sections from the laser-treated human eye were stained with the filamentous actin label phalloidin (red) and the RPE monolayer was imaged (FIG. 4A-C). The normal human RPE monolayer has a highly ordered polygonal appearance (FIG. 4A), and areas of RPE treated with the clinically relevant laser dose (0.3 mJ) showed a disrupted monolayer (FIG. 4B). When treated with suprathreshold laser dose (0.6 mJ), more distinct RPE lesions or holes are evident within the monolayer (FIG. 4C). Similar RPE lesions were present in the mouse eye, with the highly ordered RPE monolayer (FIG. 4D) disrupted by low-dose nanosecond laser treatment (0.065 mJ; FIG. 4E), and a more defined injury occurred in the high-energy group (0.13 mJ; FIG. 4F).

To investigate the RPE wound healing response, a human eye was obtained that had previously been treated with nanosecond laser on 2 separate occasions (1 mo and 1 wk) prior to exenteration. Imaging the RPE monolayer with phalloidin (red) at the region treated with nanosecond laser 1 wk prior to collection showed the presence of enlarged RPE cells on the lesion boundary, with some cells extending into the injury site (FIG. 4G). At 1 mo post-treatment, the lesion site was completely covered with enlarged RPE cells (FIG. 4H).

A similar process was evident within the mouse eye, although healing was more rapid, with a significant reduction in lesion area evident within 24 h, and the RPE monolayer was intact within 1 wk (0.065 mJ; FIG. 4J, 4I). At 7 d, cells covering the injury site were 289% larger (1112±99 vs 385±26 mm², P<0.01) than RPE cells from untreated retinae (FIG. 4I, K). As well as increasing in size, some of the RPE cells surrounding the laser injury site were also positive for the cell proliferation marker cyclin D1 (FIG. 4L), while the surrounding untreated RPE showed no evidence of cyclin D1 staining.

### Nanosecond Laser Produces a Limited Mononuclear Cell Response

Mononuclear cells such as macrophages and microglia (the resident immune cell of the CNS) are known to remove cellular debris and facilitate healing (Eter et al. 2008, Invest. Opthalmol. Vis. 49 3649; Lech et al., 2012, Mediators Inflamm). However, an uncontrolled or persistent immune response involving components such as microglial activation and/or complement cascade involvement can lead to ongoing retinal pathology, and may contribute to the etiology of AMD (Ambati et al., 2013, Nat. Rev. Immunol. 13 438). Thus, for nanosecond laser treatment to provide a clinical benefit, it ideally should not result in an overt immune response.

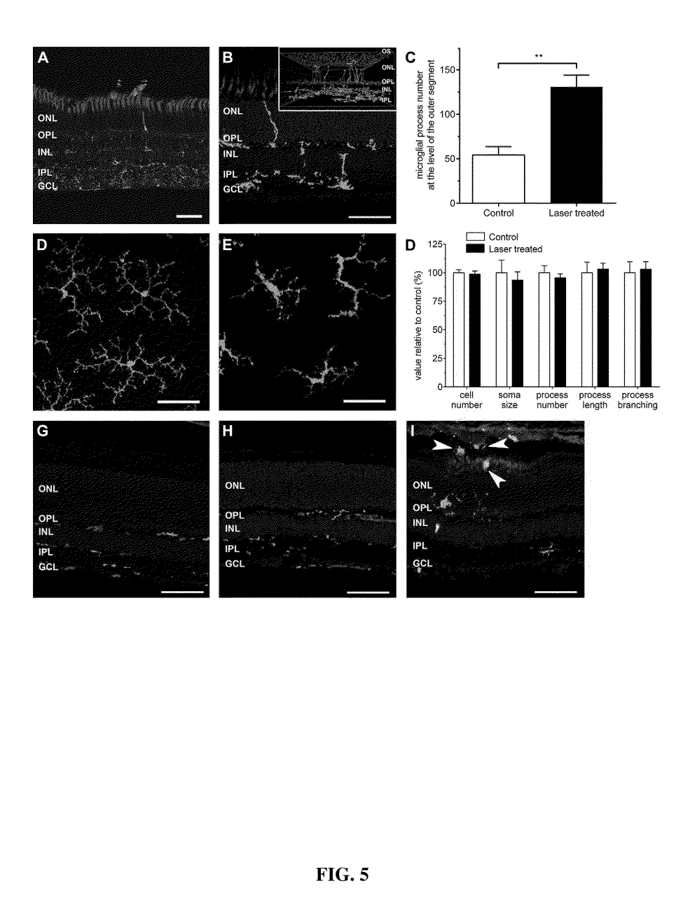

To detail the mononuclear cell response in the human retina after nanosecond laser treatment, the region encompassing the clinical dose (0.3 mJ) was labeled with the mononuclear marker, ionized calcium-binding adapter molecule 1 (green). Although the nanosecond laser did not alter retinal structure (similar to data in FIG. 3C), the cells previously observed near the tips of the outer segments (see FIG. 3C) were here identified to be of mononuclear origin (e.g., lymphocytes, monocytes, macrophages; FIG. 5A). In addition to these cells, the endogenous retinal microglia can be observed to stratify within the 2 retinal plexuses (inner plexiform layer [IPL] and OPL), and microglial processes extend through the ONL toward the laser-treated area (FIG. 5A). This microglial response seems to reflect that of the normal retina, with cell bodies found within the IPL and OPL, and processes extend throughout the retina, surveying the surrounding tissue environment.

The microglial response to nanosecond laser treatment was further investigated in the mouse retina. Similar to the human data, mouse retinal microglia were observed to send their processes toward the laser treatment site, and their cell bodies remained in the OPL (FIG. 5B, inset shows a 3-dimensional rendered image of 2 laser-treated areas). Association of microglial processes with the photoreceptor outer segments and RPE monolayer can also be observed to be limited to the laser-treated area. Although microglial processes are normally found in the outer retina in the untreated mouse retina, nanosecond laser treatment increased in the number of these processes within the ONL (FIG. 5C; 240%, 131±13 vs 54±9; P<0.01). Microglial activation, which is characterized by process retraction and increased soma size (an “amoeboid” phenotype), was characterized in the OPL directly over the laser site. The microglia in the 0.065 mJ treated retinae showed extensive process branching and normal soma sizes (FIG. 5D), and microglia directly over the high-energy laser (0.13 mJ) spot showed reduced process branching and a more “amoeboid” appearance (FIG. 5E). Quantitative assessment validated these findings, with the clinically equivalent laser dose not resulting in any of the classic signs of microglial activation (FIG. 5F, all P>0.05).

Finally, the complement response to nanosecond laser treatment was also investigated because its overexpression can lead to RPE/photoreceptor cell death (Cashman et al., 2011, Invest. Ophthalmol. Vis. Sci. 52 3436-3445). Complement factor-3 (C3) expression, which is a key component of the complement response, was similar to that found in the untreated retina and was confined to the retinal blood vessels (FIG. 5G, H). Conversely, high-dose laser treatment that resulted in extensive retinal cell death (FIG. 3H, J, K) and microglial activation (FIG. 5E), produced extensive C3 expression around the treated area (FIG. 51). Thus, although low-dose nanosecond laser treatment led to an enhanced microglial surveillance of the outer retinae, it was independent of microglial activation and complement cascade involvement.

### Nanosecond Laser Treatment Reduces BM Thickness

The previous data suggest that the nanosecond laser can reduce drusen load in patients with AMD while sparing the retina from secondary damage and overt inflammation. To investigate whether this treatment may also limit AMD-associated pathology, the ApoEnull mouse was treated with the nanosecond laser (20 laser spots, 0.065 mJ). Although no current model replicates the human disease, this mouse model is known to develop a thickened BM (Dithmar et al., 2000, Invest. Ophthalmol. Vis. Sci. 52 3436-3445), which is one of the hallmark features of AMD.

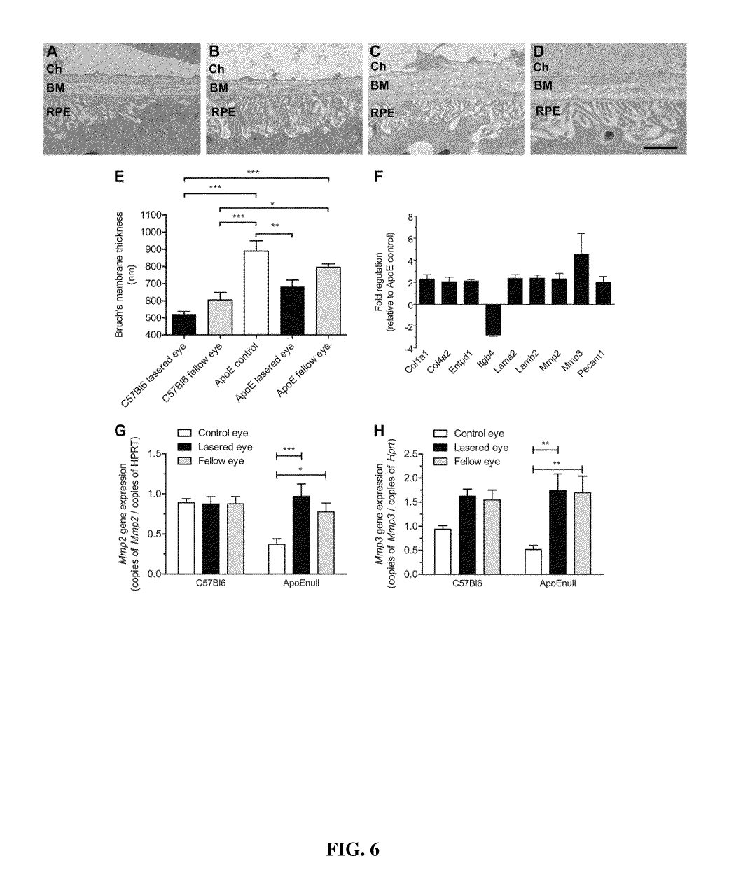

Retinal/RPE sections were isolated and imaged 3 mo post-treatment. As observed in representative control micrographs (C57BL/6J untreated fellow eye, FIG. 6A), BM separated the RPE from the choroidal blood supply (Ch) and showed no evidence of rupture or major structural damage after nanosecond laser treatment (FIG. 6A-D). Although the control tissue showed a possible minor thinning of BM after laser treatment (FIG. 6B), 13 mo ApoEnull animals, which exhibited a thickened membrane (FIG. 6C) as previously described (Dithmar et al., 2000, supra), appeared dramatically thinner after laser treatment (FIG. 6D). When quantified, BM was 47% thicker in ApoE animals compared with age-matched C57BL/6J animals (FIG. 6E, 890±60 vs 606±43 nm, n=6, P<0.001). A single nanosecond laser treatment reduced membrane thickness in the ApoEnull by 23% (FIG. 6E, 683±38 nm, P<0.01) and resulted in BM thickness not being significantly different from C57BL/6J age-matched animals (683±38 vs 606±43 nm, P>0.05). Importantly, these changes in thickness were observed across the retina and not just in the areas immediately adjacent to sites of nanosecond laser treatment. There was no significant difference between the treated and fellow eyes in the control C57BL/6J animals (FIG. 6E; 523±14 vs 606±43 nm, P>0.05), and BM thickness in the fellow eye from the laser-treated ApoEnull animals was not significantly different from either the treated eye (FIG. 6E, 795±20 vs 683±38 nm, P>0.05) or the untreated ApoEnull animals (FIG. 6E; 795±20 vs 890±60 nm, P>0.05).

ECM turnover is known to be critical for regulation of BM thickness (Kumar et al., 2010, Invest. Ophthalmol. Vis. Sci. 51 2664-2670; Kamei et al., 1999, Invest. Ophthalmol. Vis. Sci. 40 2367-2375). Total RNA was isolated from RPE-choroidal samples and a commercial PCR array was used to screen 84 ECM-related genes from the ApoEnull untreated, ApoEnull laser-treated, and ApoEnull fellow eye samples. When comparing the laser-treated ApoEnull cohort with the untreated ApoEnull, the expression of 21 genes were significantly altered (Table 2), encoding ECM proteins such as collagen (Col1a1, Col5a1, Col4a2) and laminin (Lama2, Lamb2, Lamc1), as well as ECM regulating enzymes (Mmp3, Mmp2, Timp2). Of those genes, 9 were regulated greater than 2-fold (FIG. 6F), with Mmp3 up-regulated by the greatest degree (4.5±1.9-fold), and the gene encoding the integrin subunit b4 (Itgb4) was decreased (22.8±0.1-fold). Interestingly, when the fellow eye from the ApoEnull laser-treated animal was compared with the untreated ApoEnull, 72% of the genes altered in the treated eye were also changed to a similar extent in the fellow eye (which did not receive any laser treatment; Table 3).

Gene expression of the key matrix degrading enzymes, Mmp2 and Mmp3, were subsequently quantified in all cohorts using quantitative PCR. As observed in FIG. 6G, Mmp2 expression was unchanged in the C57BL/6J laser-treated or fellow eyes when compared with the untreated control samples (treated 0.87±0.09, fellow 0.88±0.09, control 0.89±0.05, P>0.05). By comparison, untreated control ApoEnull RPE-choroidal samples exhibited a 58% reduction in Mmp2 expression when compared with control C57BL/6J samples (0.37±0.07 vs 0.89±0.05, P<0.01). When ApoEnull animals were treated with the nanosecond laser, Mmp2 expression increased in both the treated (262%, 0.97±0.16, P<0.001) and fellow (209%, 0.77±0.11, P<0.05) eyes, with expression levels restored to those observed in the C57BL/6J samples (C57BL/6J) samples vs. ApoEnull-treated and ApoEnull fellow, P>0.05). Mmp3 gene expression showed a similar response, with ApoEnull untreated levels significantly reduced compared with the C57BL/6J animals (0.52±0.09 vs 0.94±0.07, P<0.05), while levels were restored to that found in the C57BL/6J samples after laser treatment in both the treated (337%, 1.74±0.35, P<0.05) and fellow (328%, 1.69±0.35, P<0.05) eyes. Although ECM gene expression was altered by nanosecond laser treatment, the expression of genes involved in the regulation of RPE function, RPE-specific protein 65 kDa (Rpe65; photopigment recycling) and Cathepsin D (Ctsd; lysosomal function), were not altered (FIG. 7).

### Discussion

This study investigated the capacity of a low-energy, sub-threshold, nanosecond laser to reduce drusen load independent of outer retinal/RPE change in patients with AMD, detailed the cellular damage profile in human and mouse eyes, and assessed the capacity of a nanosecond laser to modulate BM thickening, one of the key pathologic changes observed in AMD. A single session of nanosecond laser treatment (12 spots) reduced drusen load in patients with AMD compared with a natural history cohort, and there was no evidence of detrimental RPE change, indicative of disease progression. Importantly, the human retinal structural studies showed no retinal damage, particularly to the light-sensitive photoreceptors, when clinically appropriate nanosecond laser dosages were applied. The human and mouse retinae exhibited similar cellular responses to the laser, with the discrete laser injury confined to the RPE monolayer, and recruitment of mononuclear cells and retinal microglial processes into the outer retina. Nanosecond laser treatment of the ApoEnull model of AMD resulted in a significant reduction in BM thickness, with an associated increase in the expression of several ECM genes, including Mmp2 and Mmp3, which returned to age-matched control levels.

Nanosecond Laser Treatment Resolves Drusen while Preserving Retinal Structure

Data from the clinical study showed that low-energy nanosecond laser reduced drusen area in 40% of treated patients with AMD after 1 yr, which was maintained out to 2 yr post-treatment (35%). Drusen regression was observed in areas adjacent to nanosecond laser-treated sites, in addition to more distant retinal areas, including the untreated fellow eye (1 yr data). This regression was greater than the spontaneous drusen resolution observed in the natural history cohort.

In addition to a reduction in drusen load, FAF imaging in areas of drusen regression showed no RPE change in the majority of laser-treated eyes (75%), with evidence of dis-ease progression (as evidence by either increased or decreased FAF) observed in only 25% of treated eyes. This is contrasted against a previous report of spontaneous drusen regression in patients with AMD, which showed 78% exhibiting an autofluorescence change indicative of disease progression. Furthermore, the human and mouse data show the repaired RPE monolayer exhibits no evidence of associated pathology, with the expression of key genes involved in RPE function (Rpe65 and CathepsinD) maintained, and Mmp-2, Mmp-3 gene expression is restored to age-matched control levels. Thus, not only does nanosecond laser treatment reduce drusen load over and above that observed in untreated patients with AMD, but RPE changes indicative of AMD progression do not appear to accompany this regression, unlike that observed in spontaneous drusen regression.

Treatment with the nanosecond laser of patients with early AMD also showed promising results with respect to progression to advanced disease. Although the natural history cohort showed almost a doubling of eyes with evidence of advanced pathology (5 eyes at 12 mo, increasing to 9 eyes at 24 mo), laser-treated eyes only showed a modest increase during the same time period (3 eyes at 12 mo, 4 eyes at 24 mo).

Although traditional thermal laser therapy reduces drusen load, the insult results in retinal damage and possibly an increased risk of choroidal neovascularization. Even other subthreshold treatments, which were thought to target the RPE and result in no retinal damage, have subsequently been shown to produce both short- and long-term damage, particularly involving the photoreceptor layer. Anatomic characterization of the human and mouse retinae after nanosecond laser treatment produced similar results, showing no evidence of neuronal damage, with the overlying photoreceptor outer segments intact. These data support our previous in vivo results (OCT), which showed occasional RPE alterations but no photoreceptor or inner retinal damage. Although nanosecond laser treatment did result in mononuclear cell accumulation and a modified retinal microglial response, the response was limited, with microglia remaining in a ramified state and no evidence of the activation of the complement cascade. Activated retinal microglia can produce numerous inflammatory mediators and the continued presence of microglia in the outer retina, together with prolonged inflammation, may be implicated in AMD. Thus, unlike thermal lasers, treatment with a nanosecond laser can reduce drusen load, without evidence of overt retinal damage or overt ocular inflammation, when used at clinically appropriate doses.

### Nanosecond Laser Thins BM by Regulating ECM Remodeling

The ApoEnull mouse, which exhibits a thickened BM (62), was treated with nanosecond laser to determine whether such treatment could reverse this key AMD-associated pathology. Control data showed that aged (13 mo) ApoEnull animals develop a thickened BM. Importantly, a single treatment session with a nanosecond laser (20 spots, 0.065 mJ) resulted in a significantly thinner membrane 3 mo after treatment. This thinning was observed across the whole tissue and not just in the vicinity of the laser spots. To our knowledge, this is the first evidence that prophylactic laser treatment reduces BM thickness, a key pathologic change in AMD.

As BM is an acellular structure, it depends upon the adjacent RPE and choroid to produce/regulate its structure. In the untreated ApoEnull animals, which showed a thickened BM, Mmp-2 (gelatinase) and Mmp-3 (stromelysin-1) gene expression was significantly reduced in RPE-choroidal samples. As both Mmp-2 and Mmp-3 are expressed by the RPE, are found within BM, and degrade ECM components found in the membrane, decreased Mmp expression would likely increase membrane thickness and would explain the increased BM thickness. When the ApoEnull animals were treated with nanosecond laser, Mmp-2 and Mmp-3 expression was restored to those of age-matched controls. A previous study also reported increased MMP-9 levels after laser treatment (Zhang et al., 2012, Invest. Ophthalmol. Vis. Sci. 53 2928-2937); however, this was not found in our PCR array data. Rather Mmp-3 expression was increased, which is known to be important for activation of other Mmps, such as Mmp-1, -7, and -9. Importantly, unlike the in vitro studies that followed Mmp expression out for a maximum of 14d, this study showed increased Mmp2 and Mmp3 expression 3 mo after laser treatment, suggesting a prolonged laser-induced effect.

In addition to the changes in Mmp gene expression, the gene array data also showed nanosecond laser treatment increased the expression of several ECM genes such as collagen (Col1a1, 5a1, 4a2), laminin (Lama2, Lamb2, Lamc1), and components of elastic fibers (Emilin1), in addition to several integrin subunits. Several of these components show an age-dependent reduction in BM directly adjacent to the RPE, leading to disruption of BM-RPE attachment. This alteration was particularly found in areas overlying drusen deposits. Thus, a laser-induced increase in these ECM proteins, in addition to increased integrin expression, may improve the attachment of the overlying RPE to BM, inhibit the RPE detachment, and slow the atrophic process observed in AMD. Thus, nanosecond laser treatment appears capable of reversing some of the key age-related alterations in BM and RPE attachment that predisposes individuals to AMD.

Interestingly, our clinical cohort showed a reduction in drusen within the fellow untreated eyes, and the mouse gene expression data also showed evidence of a nanosecond laser-induced fellow eye effect, with RPE-choroidal expression of Mmp2, Mmp3, and several other ECM genes increased to a similar degree to that observed in the treated eye. Such binocular effects have been reported previously in studies using monocular treatment paradigms, including 1 study involving the nanosecond laser (44, 53).

Despite this, the fellow eye effect was not observed with respect to BM, for although there was a mean decrease in thickness in the fellow eye, it was not significant. This apparent disconnect between the matrix gene regulation and membrane thickness may reflect a difference in the temporal nature of the laser-induced effect between the treated and fellow eyes, with longer post-treatment time required to thin BM in the fellow eye. It is also possible that a different set of genes were altered in the fellow eyes, compared with the treated eye, resulting in slightly modified response. Nevertheless, given the reduction in drusen within the fellow untreated eyes of our clinical cohort and the nanosecond laser-induced fellow eye effects observed in our mouse model, this binocular effect requires further investigation.

### The Mechanism for Drusen Resolution

Drusen regression following prophylactic laser treatment has been reported over many years; however, a mechanism for clearance of drusen remains unclear. Similarly, whether such drusen clearance reflects a slowing of disease progression is unknown. Our human and mouse data suggest an important role for RPE-choroidal cells and BM in facilitating drusen removal.

Our data show that nanosecond laser treatment results in a specific alteration in ECM-regulating factors within the RPE-choroidal cells. This laser-induced RPE alteration has previously been hypothesized by Frennesson and Nilsson (Frennesson & Nilsson, 1998, Br. J. Ophthalmol. 82 1169-1174); however, they suggested the newly formed RPE could better phagocytose drusen material. By contrast, our data support a specific matrix-mediated effect rather than a change in RPE metabolism, because the expression of cathepsin-D and RPE65 (markers of RPE lysosomal action and opsin recycling; FIG. 7) were not altered by the laser. As BM relies on RPE-choroidal cells to mediate its structure, this altered matrix regulation would likely lead to the thinned membrane observed in the laser-treated ApoEnull animals. In addition to BM thickness, the movement of molecules through the membrane is critical to the influx of important nutrients and the removal of waste products. Previous work has shown an age-related decline in the transport of molecules across BM, the accumulation of lipid deposits, and drusen-based material and the progression toward AMD Although this study provides no direct assessment of the efficacy of nanosecond laser to improve the conductivity of the membrane, previous work has shown a reduction in BM hydraulic conductivity after Mmp exposure in vitro. Therefore, the thinned BM observed in this study, in conjunction with an increased Mmp expression and improved RPE-membrane interaction, is likely to reduce outflow resistance and facilitate removal of drusen, as well as improve overall RPE health and function. In addition to direct removal of drusen material via BM, the thinned membrane may also facilitate additional immune-mediated removal of accumulated drusen.

This study has shown that prophylactic nanosecond laser treatment can bring about the resolution of drusen in those with AMD, while maintaining normal retinal structure. This is critically important for the light-sensitive photoreceptors that are often damaged in thermal ophthalmic laser treatments. As the human and mouse retina show similar responses to nanosecond laser treatment, the thinning of BM and increase in several membrane constituents that improve RPE-membrane attachment may indicate this laser has the potential to improve membrane structure and function in humans. These improvements are crucial to the health of the retina, as reduced transport across BM is integral to the development of AMD. This study demonstrates biologically relevant pathways by which the nanosecond laser improves the pathologic changes known to be implicated in the development of AMD and as such provides a potentially viable intervention for all those at risk of vision loss from its devastating consequences.

### Example 2

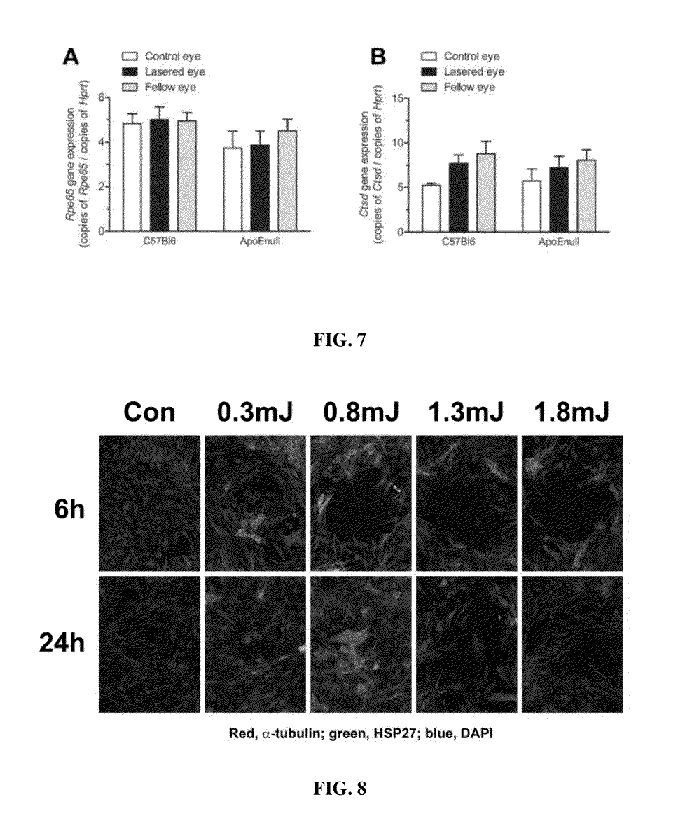

Referring to FIG. 8, the eyes of mice were irradiated with either 0.3 mJ, 0.8 mJ, 1.3 mJ or 1.8 mJ energy levels which resulted in physical ablation of cells, as shown by detecting expression of the pan-cellular marker α-tubulin, which labels all cells consistently. At the time intervals post-irradiation measured (6 hr and 24 hr), cells began to grow back into the killed-zones together with induction of apoptosis (as seen by small, intense blue nuclei compared with normal more diffuse nuclei). There was also induction of stress-response proteins around the lased area (i.e. HSP27).

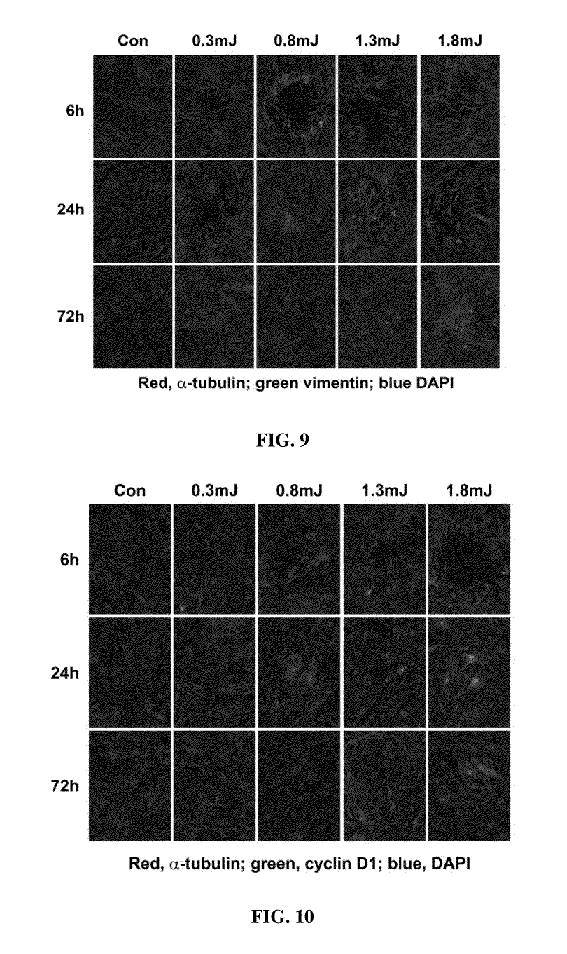

Referring now to FIG. 9, after laser irradiation with either 0.3 mJ, 0.8 mJ, 1.3 mJ or 1.8 mJ energy levels, cells were labelled with α-tubulin antibodies (as a pan-cellular-marker) and also anti-vimentin antibodies. Vimentin is not usually expressed by epithelial cells, but when they undergo various forms of stress they dedifferentiate and undergo “epithelial-mesenchymal transition”; vimentin being a classic example of a mesenchymal cell label. In and around laser spots in the cultures, we observed cells expressing vimentin, sometimes even without expressing tubulin and often within 6 hrs of laser. These cells are true de-differentiated mesenchymal cells.

Cyclin D1 is a marker of cell proliferation and is induced in cells around the edge and within the laser-treated zones of the eye as time increases post laser ablation (FIG. 10).

In summary, these data show that instead of the RPE cells just growing or dividing back to fill laser lesion sites, they are stimulated to change their phenotype, divide/migrate and then redifferentiate to RPE. By altering their phenotype to mesenchymal cells (which are effectively de-differentiated cells, they are able to move or divide into the right place, fill the gap completely and then re-establish their tight junctions and polarity. This is a novel finding that RPE can utilize the mechanism of epithelial-mesenchymal transition for wound-healing. This implies an inherent plasticity of the RPE.

Firstly, expression of stress markers (i.e such as HSP27), likely signalled by loss of contact inhibition followed by dedifferentiation of RPE to mesenchymal cells. This is followed by proliferation of new mesenchymal cells into the ablated zones and finally an opposite reaction, “mesenchymal-epithelial transition”, where differentiated RPE cells are re-formed and the monolayer reverts to its pre-lasered state.

Persons skilled in the field will be aware of specific elements that may be substituted for one or more of the elements described for the preferred embodiment without departing from the spirit and scope of the invention. Throughout the specification the aim has been to describe the invention without limiting the invention to any one particular combination of preferred features or embodiment.

All computer programs, algorithms, patent and scientific literature referred to herein is incorporated herein by reference.

TABLE 1
The effect of nanosecond laser treatment on the development of advanced
AMD-related disease

| Time post- | Natural history | Nanosecond | Fellow untreated |
|---|---|---|---|
| treatment (mo) | eyes | laser-treated eyes | eyes |
| 12 | 6/116 (5%) | 3/50 (6%) | 2/50 (4%) |
| 24 | 10/116 (9%) | 4/50 (8%) | 3/50 (6%) |

The efficacy of nanosecond laser treatment was assessed in a pilot clinical study involving 50 individuals with intermediate AMD (drusen >125 mm) and compared with a natural history cohort (58 individuals) with the same inclusion criteria. All underwent baseline visual assessment and were free of signs of advanced AMD (geographic atrophy/choroidal neovascularization). Data showing the progression to advanced forms of AMD are presented, with relevant percentages shown in parentheses. Of those that had progressed to late stage disease, all in the treated group showed evidence of geographic atrophy, and the natural history cohort exhibited geographic atrophy or choroidal neovascularization (3/3 at 12 mo, 7/3 at 24 mo).

TABLE 2
Regulation of extracellular matrix genes in the ApoEnull treated RPE/
choroid following nanosecond laser treatment.

| ECM | Gene | Fold | |
|---|---|---|---|
| constituent | name | Regulation | Description |
| structural | Col1a1 | 2.3 | Alpha 1 chain, type I collagen |
| protein | | | |
| | Col5a1 | 1.8 | alpha 1 chain, type V collagen |
| | **Col4a2 | 2.1 | alpha 2 subunit, type IV collagen |
| | Lama2 | 2.3 | alpha 2 chain, laminin 2 and |
| | | | laminin 4 |
| | Lamb2 | 2.4 | beta 2 chain, laminin |
| | Lamc1 | 1.5 | gamma 1 chain, laminin |
| | Emilin | 1.7 | elastin microfibril interfacer 1 |
| regulating | Adamts2 | 1.8 | A disintegrin and metalloproteinase |
| enzyme | | | with thrombospondin motifs 2 |
| | Mmp2 | 2.3 | Matrix metalloproteinase 2 |
| | Mmp3 | 4.3 | Matrix metalloproteinase 3 |
| | Timp2 | 1.3 | Tissue inhibitor metalloproteinase 2 |
| | Entpd1 | 2.1 | Ectonucleoside triphosphate |
| | | | diphosphohydrolase 1 |
| cell-cell, | Pecam1 | 2.0 | Platelet endothelial cell adhesion |
| cell-matrix | | | molecule |
| interaction | | | |
| | Spp1 | 1.9 | Secreted phosphoprotein 1 |
| | Thbs2 | 1.8 | Thrombospondin-2 |
| | Itgam | 1.8 | integrin alpha M chain |
| | **Itga4 | 1.6 | integrin alpha 4 chain |
| | **Itgax | 1.7 | integrin alpha X chain |
| | **Itgb1 | 1.5 | integrin beta 1 chain |
| | **Itgb3 | 1.6 | integrin beta 3 chain |
| | **Itgb4 | 2.7 | integrin beta 4 chain |

A commercial extracellular matrix PCR array was used to screen 84 ECM-related genes in RPE/choroidal samples taken from the treated eyes of nanosecond laser treated animals. Samples were pooled and 3 independent arrays performed. Data is relative to untreated control ApoEnull samples and all highlighted gene changes were statistically significant (P < 0.05, unpaired t-test).
**highlights genes that were only regulated in the treated ApoEnull eye.

TABLE 3
Regulation of extracellular matrix genes in the ApoEnull fellow RPE/
choroid following nanosecond laser treatment.

| ECM | Gene | Fold | |
|---|---|---|---|
| constituent | name | Regulation | Description |
| structural | Col1a1 | 1.9875 | Alpha 1 chain, type I collagen |
| protein | | | |
| | Col5a1 | 1.6046 | alpha 1 chain, type V collagen |
| | **Col4a1 | 1.6876 | alpha 1 subunit, type IV collagen |
| | **Col6a1 | 1.6046 | alpha 1 subunit, type VI collagen |
| | Lama2 | 1.9592 | alpha 2 chain, laminin 2 and |
| | | | laminin 4 |
| | Lamb2 | 1.8109 | beta 2 chain, laminin |
| | Lamc1 | 1.6212 | gamma 1 chain, laminin |
| | Emilin | 1.7116 | elastin microfibril interfacer 1 |
| regulating | **Adamts1 | 1.6551 | A disintegrin and metalloproteinase |
| enzyme | | | with thrombospondin motifs 1 |
| | Adamts2 | 1.8033 | A disintegrin and metalloproteinase |
| | | | with thrombospondin motifs 2 |
| | Mmp2 | 2.1962 | Matrix metalloproteinase 2 |
| | Mmp3 | 4.092 | Matrix metalloproteinase 3 |
| | Timp2 | 1.2592 | Tissue inhibitor |
| | | | metalloproteinase 2 |
| | Entpd1 | 1.8458 | Ectonucleoside triphosphate |
| | | | diphosphohydrolase 1 |
| cell-cell, | Pecam1 | 1.9119 | Platelet endothelial cell adhesion |
| cell-matrix | | | molecule |
| interaction | | | |
| | Spp1 | 1.7782 | Secreted phosphoprotein 1 |
| | Thbs2 | 1.6487 | Thrombospondin-2 |
| | Itgam | −1.9149 | integrin alpha M chain |
| | **Itgb5 | 1.897 | integrin beta 5 chain |
| | **Icam1 | 1.778 | Intercellular adhesion molecule 1 |
| | **Syt1 | −1.7509 | Synaptotagmin-1 |

A commercial extracellular matrix PCR array was used to screen 84 ECM-related genes in RPE/choroidal samples taken from the fellow eyes of nanosecond laser treated animals. Samples were pooled and 3 independent arrays performed. Data is relative to untreated control ApoEnull samples and all highlighted gene changes were statistically significant (P < 0.05, unpaired t-test).
**highlights genes that were only regulated in the fellow ApoEnull eye.

1. A method of determining the effect of a laser treatment of a disease, disorder or condition of the eye or eyes, said method including the step of detecting one or more biological responses by the eye or eyes subsequent to laser treatment of the eye or eyes to thereby determine the effect of the laser treatment.

2. The method of claim 1, wherein the one or more biological responses by the eye or eyes are responses by cells, tissues and/or molecules of the eye or eyes that indicate, are associated with, or are functionally linked to, the effect of the laser treatment.

3. The method of claim 2, which includes the step of detecting or measuring a change in the expression of one or more genes or encoded proteins after laser treatment.

4. The method of claim 3, wherein the genes are selected from the group consisting of: one or more cytoskeletal genes; one or more cell cycle genes;
and one or more extracellular matrix genes.

5. The method of claim 4, wherein: the cytoskeletal gene encodes vimentin; the encode Mmp2 and/or Mmp3; the cell cycle gene encodes Cyclin D1; and the extracellular matrix genes encode matrix metalloproteinases, collagen, laminin, elastic fiber proteins and/or one or more integrin subunits.

6. The method of claim 2 which includes the step of detecting or measuring cell differentiation, cell death and/or cell proliferation after laser treatment.

7. The method of claim 1, wherein the disease, disorder or condition is a degenerative disease, disorder or condition of the eye or eyes.

8. The method of claim 1, wherein the disease, disorder or condition is a macular or retinal disease, disorder or condition.

9. The method of claim 1, wherein the disease disorder or condition is age-related macular degeneration (AMD), Diabetic Macular Edema (DME), glaucoma, retinitis pigmentosa, atrophy of the neuro-retina, stargardt disease, central serous retinopathy, choroidal neovascularization (CNV) or cystoid macular edema.

10. The method of claim 1 which further includes measuring thinning of Bruch's membrane and/or measurement of drusen levels as an indication of the effect of the treatment.


SEQUENCE LISTING (8 sequences)

SEQ ID NO: 1 - DNA, 20 bp, Artificial sequence
  PCR primer
gtcgcccctaaaacagacaa

SEQ ID NO: 2 - DNA, 20 bp, Artificial sequence
  PCR primer
ggtctcgatggtgttctggt

SEQ ID NO: 3 - DNA, 20 bp, Artificial sequence
  PCR primer
cagacttgtcccgtttccat

SEQ ID NO: 4 - DNA, 20 bp, Artificial sequence
  PCR primer
ggtgctgactgcatcaaaga

SEQ ID NO: 5 - DNA, 22 bp, Artificial sequence
  PCR primer
cctaagatgagcgcaagttgaa

SEQ ID NO: 6 - DNA, 24 bp, Artificial sequence
  PCR primer
ccacaggactagaacacctgctaa

SEQ ID NO: 7 - DNA, 20 bp, Artificial sequence
  PCR primer
tgtgtccgtcgtggatctga

SEQ ID NO: 8 - DNA, 21 bp, Artificial sequence
  PCR primer
ttgctgttgaagtcgcaggag